(19) United States
(12) Patent Application Publication
(10) Pub. No.: US 2018/0156781 A1
(43) Pub. Date: Jun. 7, 2018

(54) MODELS, METHODS AND COMPOSITIONS FOR TREATING INFLAMMATORY BOWEL DISEASE

(71) Applicant: CEDARS-SINAI MEDICAL CENTER, Los Angeles, CA (US)

(72) Inventors: David Q. SHIH, La Crescenta, CA (US); Stephan R. TARGAN, Santa Monica, CA (US); Yoshitake KANAZAWA, Morioka (JP); Janine BILSBOROUGH, Simi Valley, CA (US)

(21) Appl. No.: 15/571,566

(22) PCT Filed: May 12, 2016
(86) PCT No.: PCT/US2016/032180

§ 371 Date: Nov. 3, 2017

Related U.S. Application Data
(60) Provisional application No. 62/162,559, filed on May 15, 2015.

(51) Int. Cl. G01N 33/50; A61K 38/19; A61P 1/04; A61P 1/16; A61P 1/06; A61K 48/00

(52) U.S. Cl. CPC G01N 33/5041; A61K 38/191; A61P 1/04; C12N 2015/8536; A61P 1/06; A61K 48/0066; A01K 2267/0368; A61P 1/16

(57) ABSTRACT

The invention provides models of various conditions including but not limited to intestinal inflammation and/or fibrosis, inflammatory bowel disease, colitis, acute colitis, and chronic colitis, and methods of using such models for designing, screening and developing therapeutics for those conditions. The invention also provides methods, compositions, and kits for treating those conditions.

### FIELD OF THE INVENTION

The invention relates to various models, systems, compositions, methods and kits for treating a condition and for designing, screening and developing therapeutics for a condition. The condition includes but is not limited to intestinal inflammation and/or fibrosis, inflammatory bowel disease, colitis, acute colitis, and chronic colitis.

### BACKGROUND

All publications, patents, patent application, and literature references cited herein are hereby incorporated by reference in their entirety to the same extent as if each individual publication, patent, patent application, or literature reference was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Crohn's disease (CD) and ulcerative colitis (UC), the two common forms of inflammatory bowel disease (IBD), are chronic, relapsing inflammatory disorders of the gastrointestinal tract. Each has a peak age of onset in the second to fourth decades of life and prevalences in European ancestry populations that average approximately 100-150 per 100,000 (D. K. Podolsky, N Engl J Med 347, 417 (2002); E. V. Loftus, Jr., Gastroenterology 126, 1504 (2004)). Although the precise etiology of IBD remains to be elucidated, a widely accepted hypothesis is that ubiquitous, commensal intestinal bacteria trigger an inappropriate, overactive, and ongoing mucosal immune response that mediates intestinal tissue damage in genetically susceptible individuals (D. K. Podolsky, N Engl J Med 347, 417 (2002)).

For treating IBD, several medications have been made to target immune pathways other than TL1A. However, these existing medications are effective only about 50% of time, and oftentimes become ineffective over time. While TL1A and DR3 are in the same signaling pathway, we demonstrated in two different animal models that blocking TL1A but not DR3 has a beneficial effect. Thus in various embodiments of the present invention, we provide a new treatment approach that targets TL1A to treat IBD patients, including but not limited to those not responding to the existing medications or lost response to the existing medications.

### SUMMARY OF THE INVENTION

The following embodiments and aspects thereof are described and illustrated in conjunction with systems, compositions and methods which are meant to be exemplary and illustrative, not limiting in scope.

Various embodiments of the present invention provide a model of a condition. In various embodiments, the condition is intestinal inflammation and/or fibrosis, inflammatory bowel disease, and/or chronic colitis. In some embodiments, the model is a quantity of cells overexpressing or constitutively expressing TL1A and/or DR3. In some embodiments, the model is an animal that has been injected with a quantity of cells overexpressing or constitutively expressing TL1A and/or DR3. In some embodiments, the model is a transgenic animal with a transgene overexpressing or constitutively expressing TL1A and/or DR3. In some embodiments, the model is a gene knockout animal with TL1A and/or DR3 gene knocked out. In various embodiments, the model exhibits fibrostenosis, inflammation in the gastrointestinal (GI) tract, weight loss, and/or an increase in disease-activity index. In various embodiments, the model has DSS-induced colitis. In various embodiments, the model is colitis induced by adoptive transfer.

Various embodiments of the present invention provide a method of identifying an agent as being therapeutic to a condition. The method may comprise: providing a model of the condition; administering the agent to the model; detecting one or more changes in the model to determine if the agent inhibits TL1A activity; and identifying the agent that is determined to inhibit TL1A activity as being therapeutic to the condition. In various embodiments, the condition is intestinal inflammation and/or fibrosis, inflammatory bowel disease, and/or chronic colitis.

Various embodiments of the present invention provide a method of treating, preventing, reducing the likelihood of having, reducing the severity of and/or slowing the progression of a condition in a subject. The method may comprise: providing an agent that inhibits TL1A activity; and administering a therapeutically effective amount of the agent to the subject, thereby treating, preventing, reducing the likelihood of having, reducing the severity of and/or slowing the progression of the condition in the subject. In various embodiments, the condition is intestinal inflammation and/or fibrosis, inflammatory bowel disease, and/or chronic colitis. In various embodiments, the agent is an anti-TL1A antibody or a functional fragment thereof.

Various embodiments of the present invention provide a composition. The composition may comprise: an agent that inhibits TL1A activity. In various embodiments, the agent is an anti-TL1A antibody or a functional fragment thereof.

Various embodiments of the present invention provide a kit for treating, preventing, reducing the severity of and/or slowing the progression of a condition in a subject. The kit may comprise: a quantity of an agent that inhibits TL1A activity; and instructions for using the agent to treat, prevent, reduce the likelihood of having, reduce the severity of and/or slow the progression of the condition in the subject. In various embodiments, the agent is an anti-TL1A antibody or a functional fragment thereof.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 illustrates, in accordance with various embodiments of the invention, TL1A-DR3 signaling. TL1A is a TNF superfamily member and the only known receptor is DR3. TL1A is pathogenic in several autoimmune models and has a pleiotropic effect such as apoptosis, proliferation, fibrosis and immune activation. Either Tl1a or Dr3 deficiency is protective in autoimmune disease models. Deficiencies in either TL1A or DR3 have been found to prevent autoimmune disease in several models like experimental autoimmune encephalomyelitis (EAE), allergic lung inflammation (ALI) and collagen/antigen-induced arthritis model (CIA or AIA). There are also evidences that Tl1a and Dr3 are involved in intestinal homeostasis and IBD.

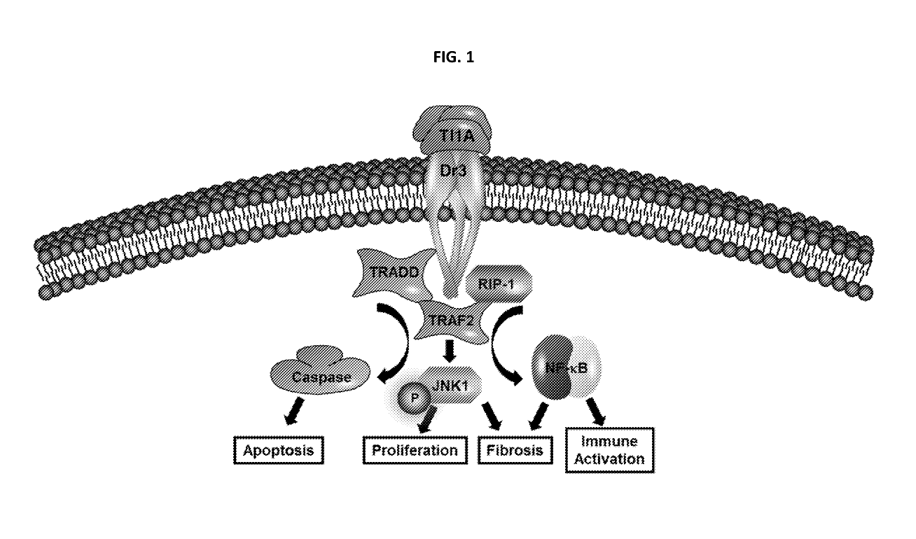

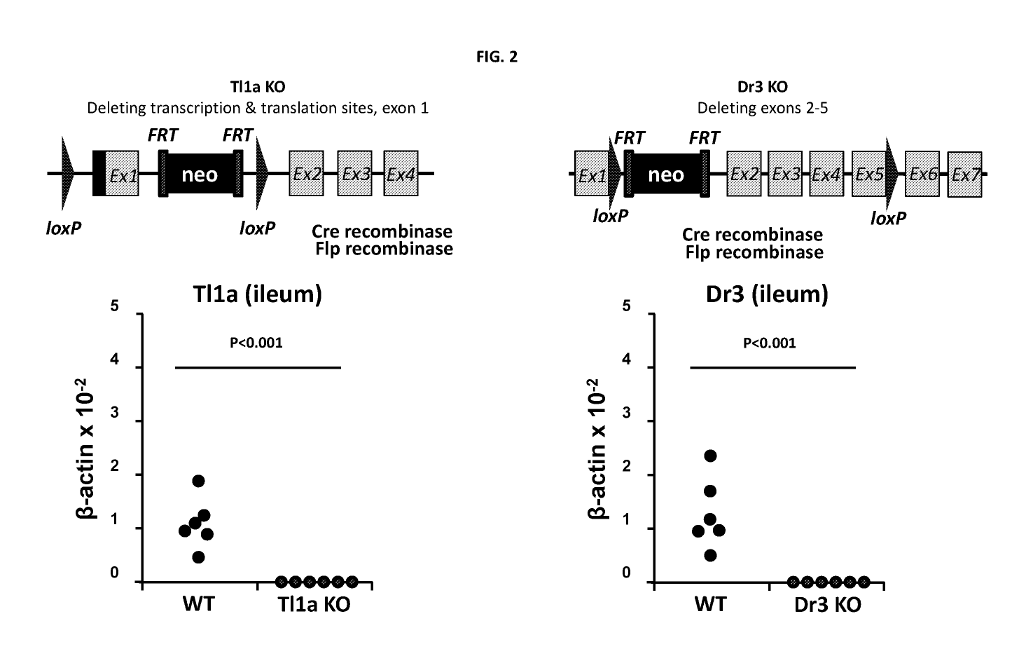

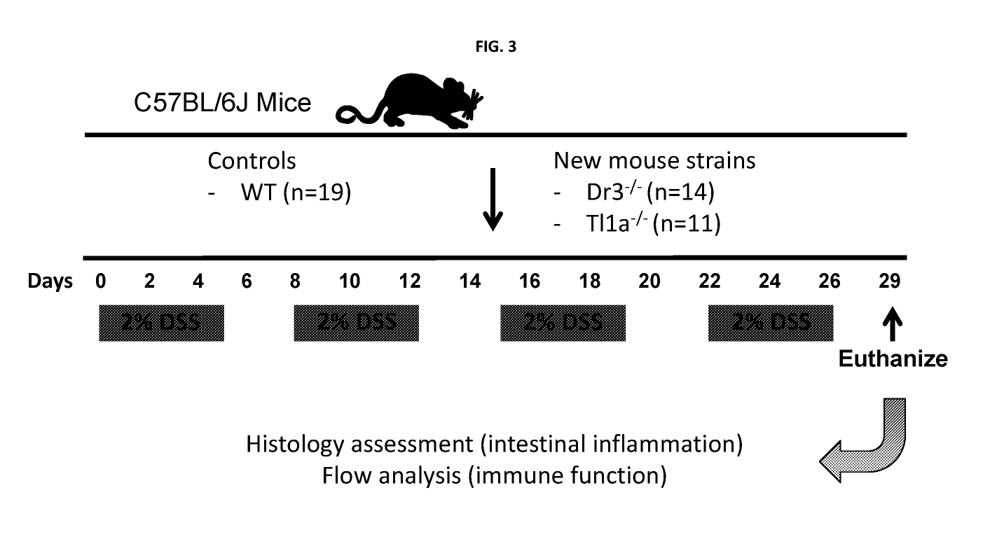

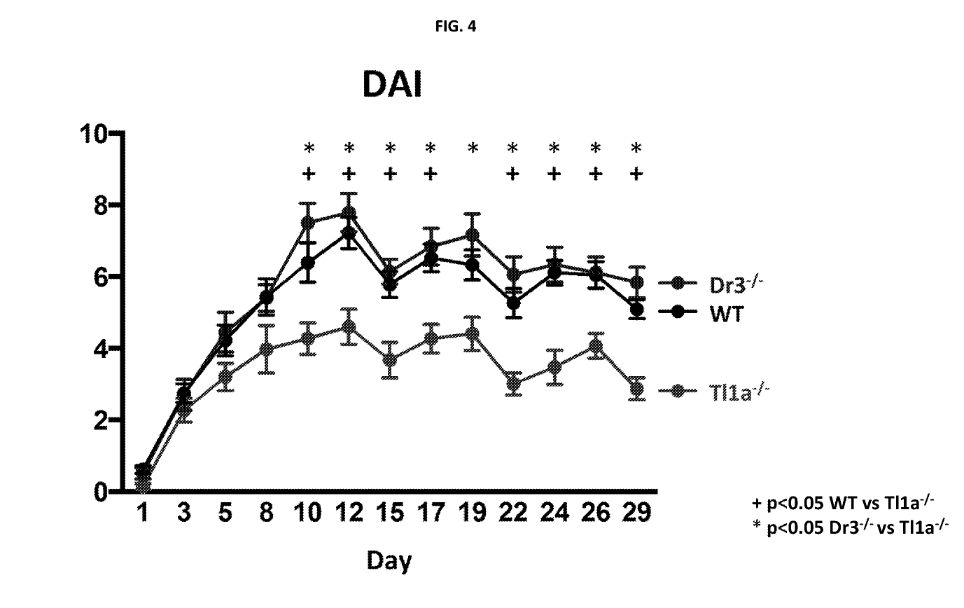

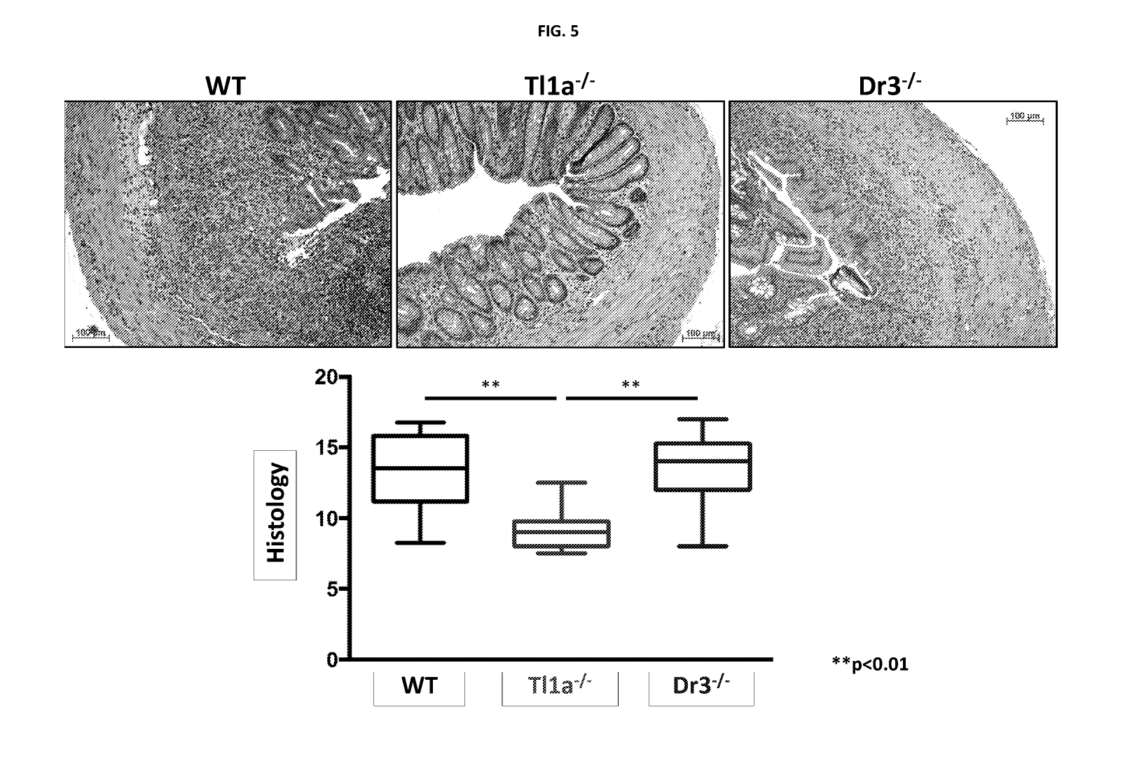

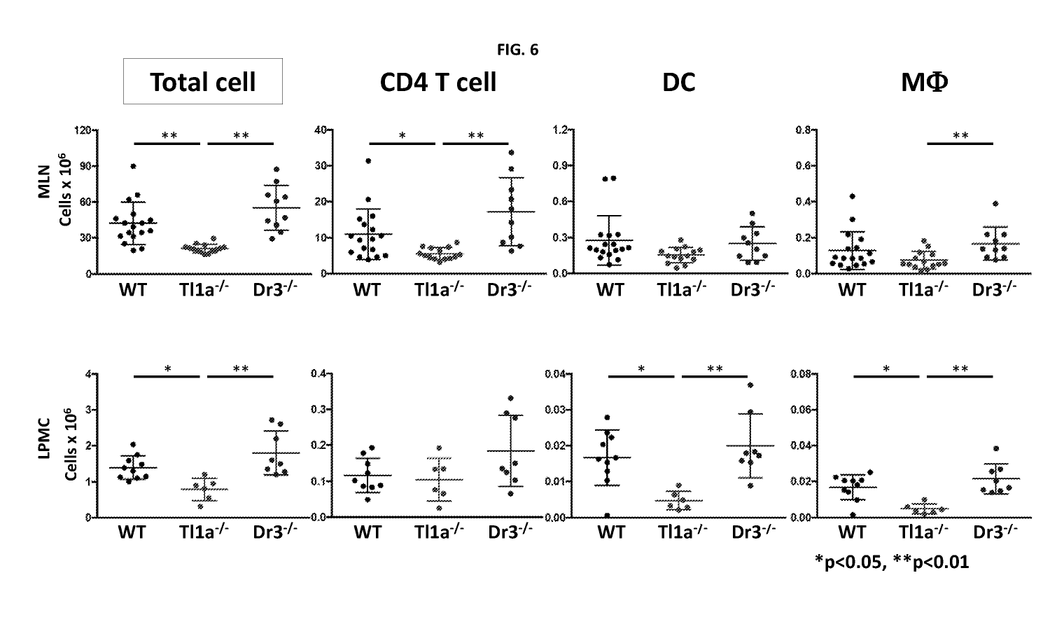

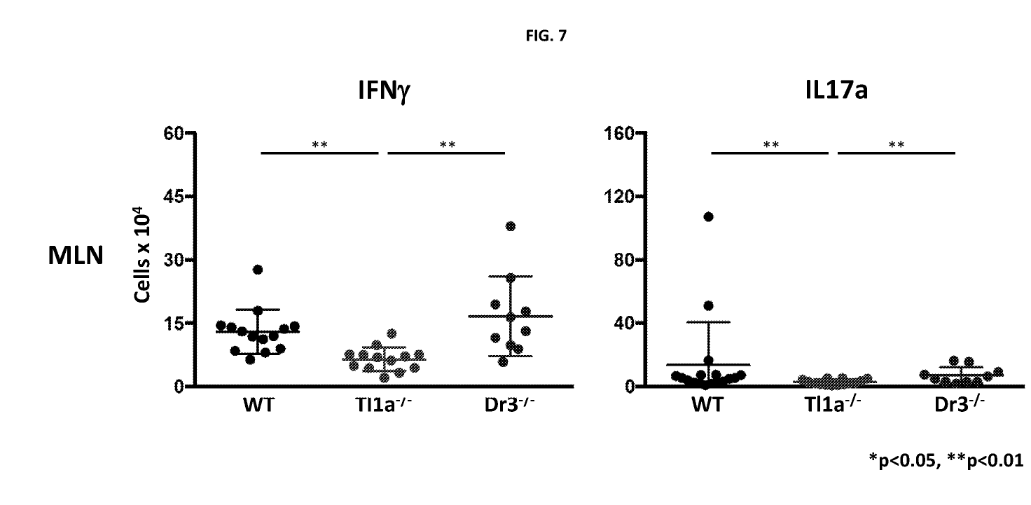

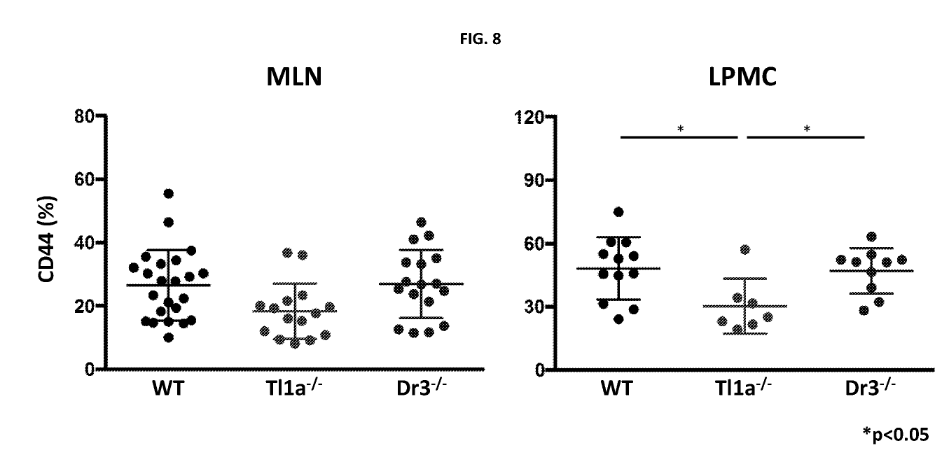

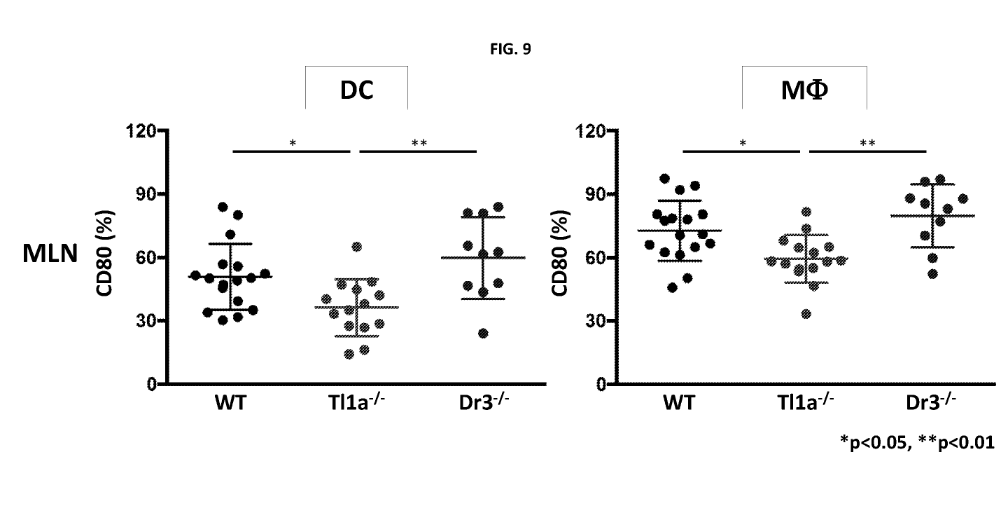

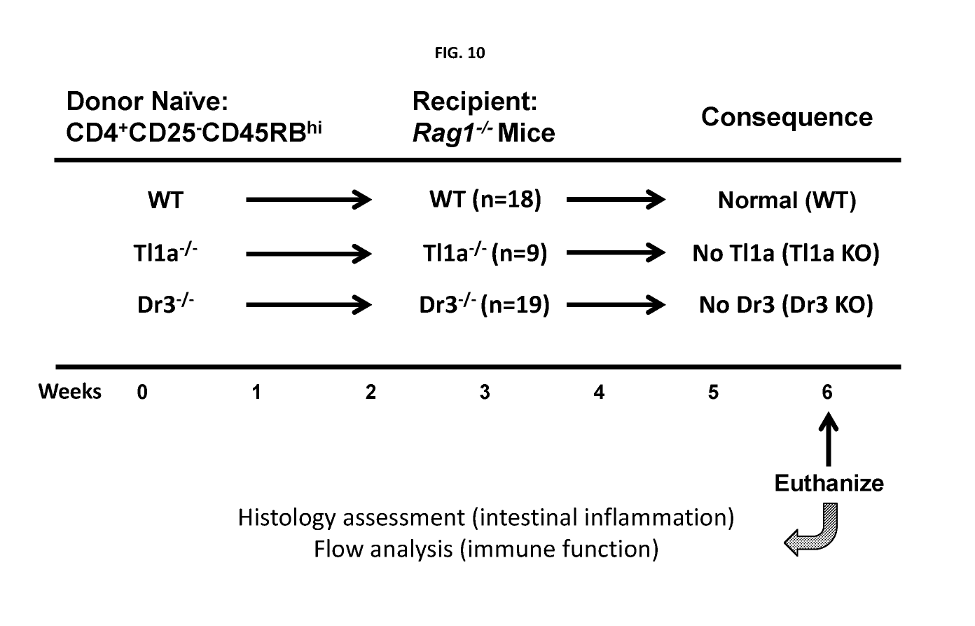

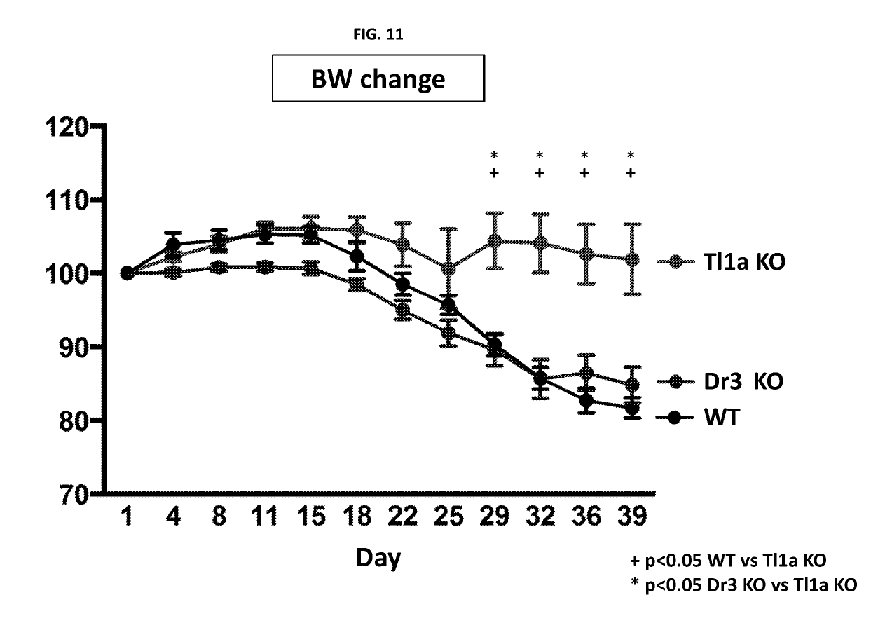

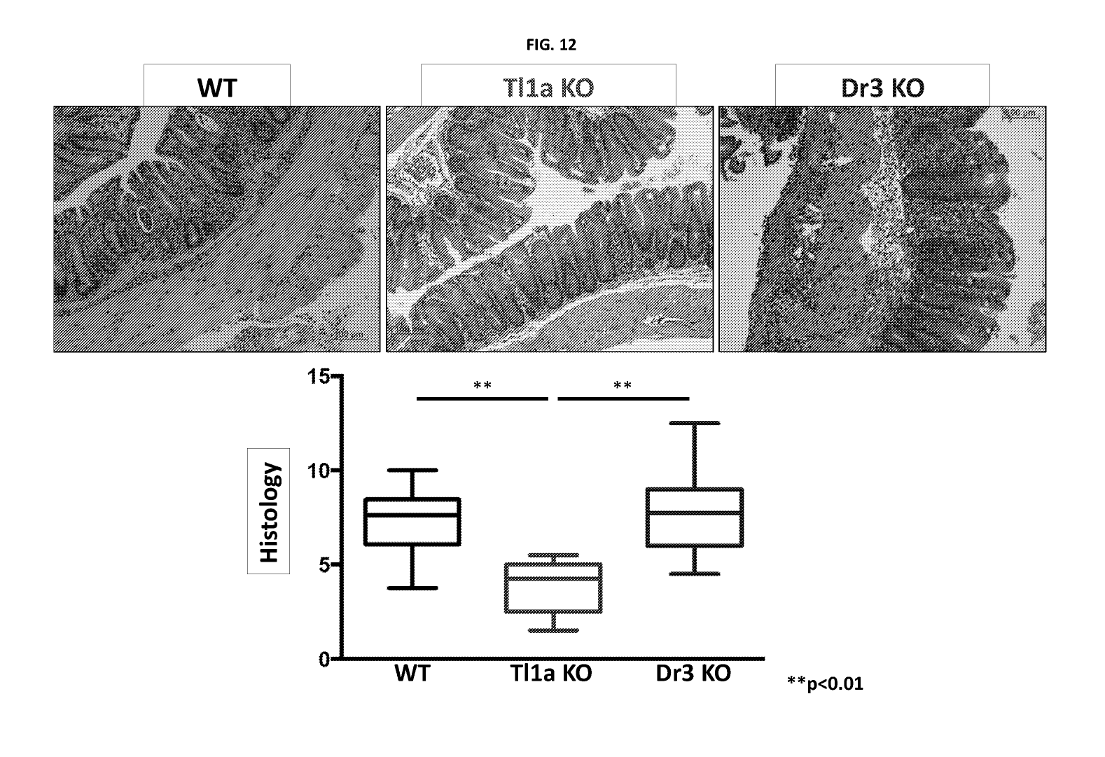

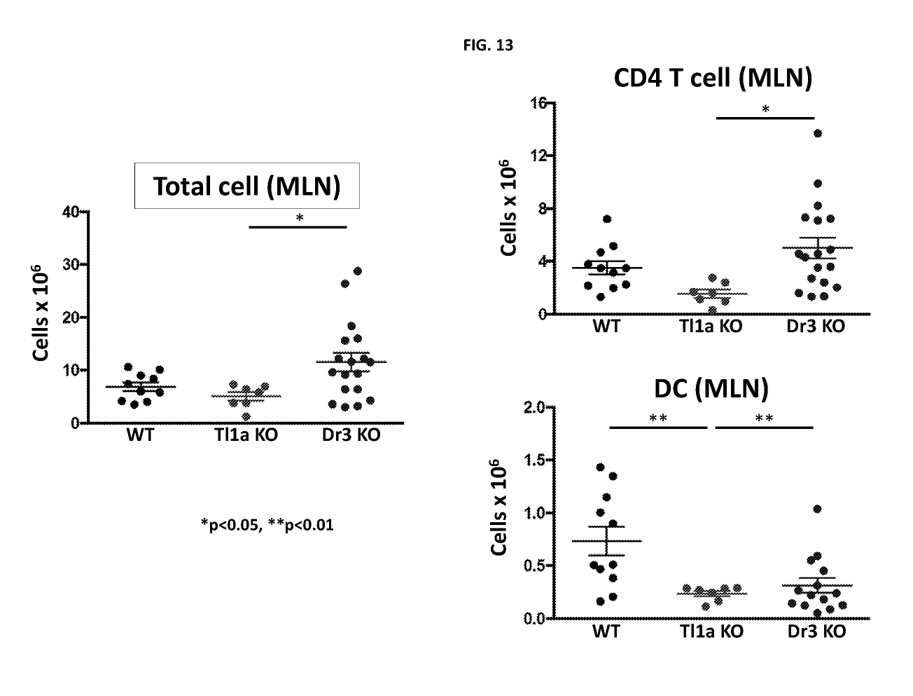

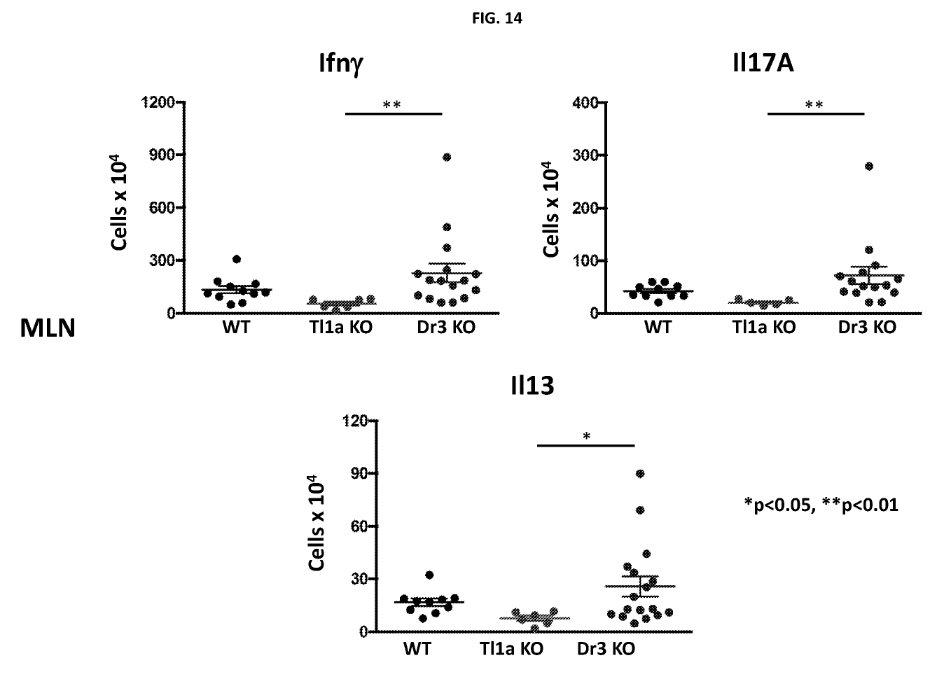

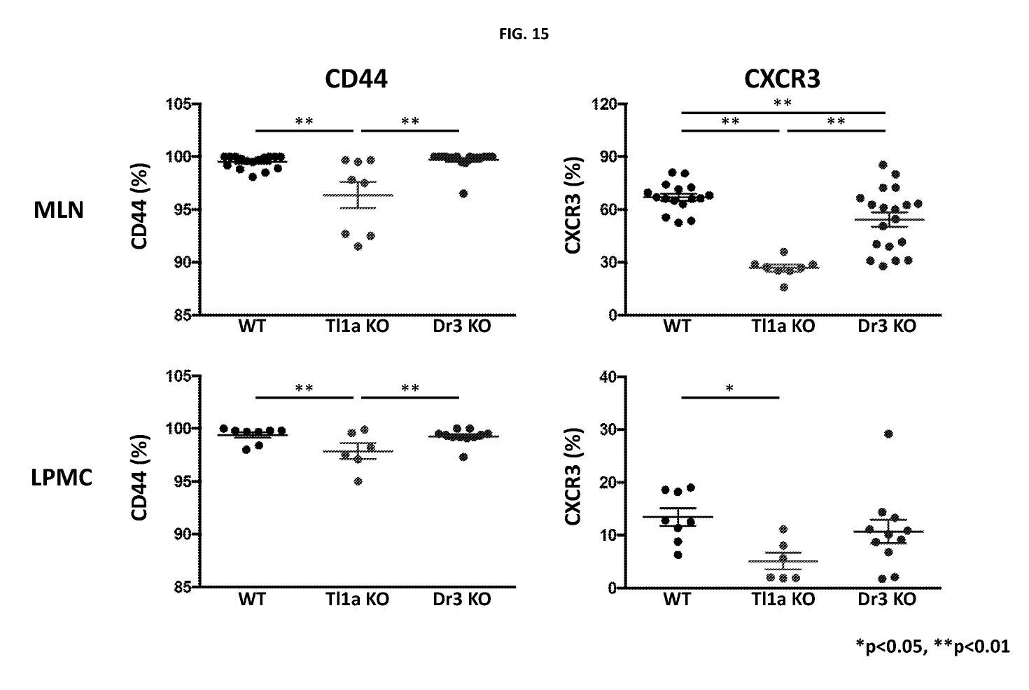

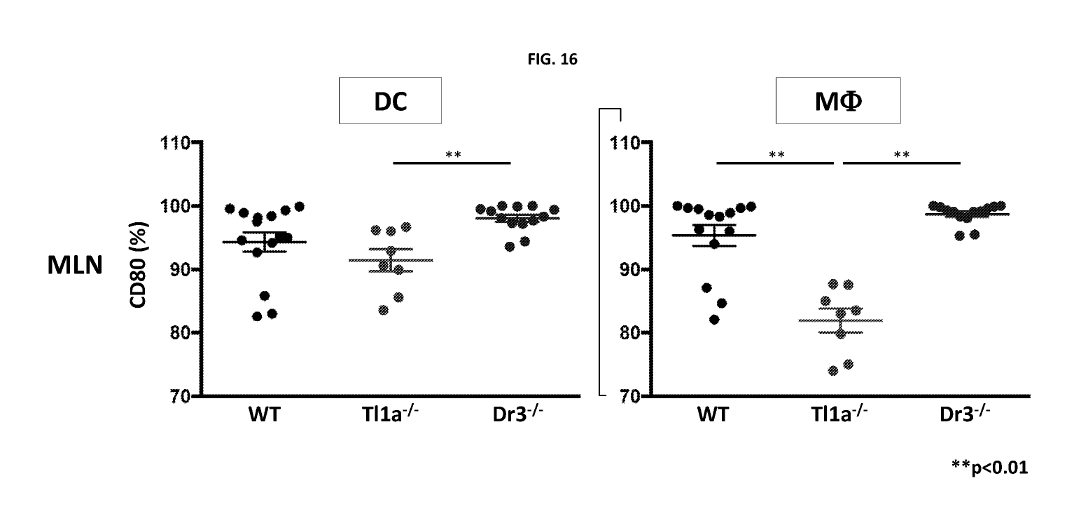

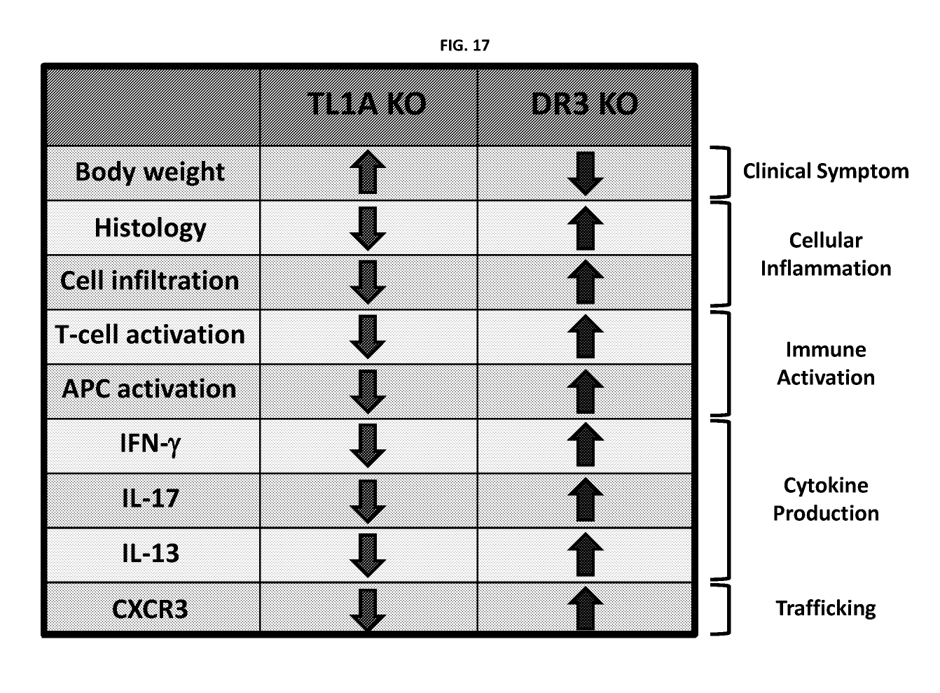

TL1A and DR3 is likely pathogenic in intestinal inflammation. In the intestine, TL1A has been found to promote Th-1, -2, -9, -17, ILC2 and ILC3. Genetic studies have also found that TNFSF15 polymorphisms are associated with increased TL1A expression and IBD. It is also shown that blocking TL1A antibody can reverse two murine colitis models. Mice with constitutive Tl1a expression develop spontaneous ileitis and worsened ileo-cecal inflammation in colitis models. However, effects of TL1A and DR3 deficiency in colitis models have not been really studied. As such, we investigated whether both TL1A and DR3 deficiency are protective in murine models of chronic colitis similar to other non-gut autoimmune disease models.

FIG. 2 illustrates, in accordance with various embodiments of the invention, generation of Tl1a and Dr3 KO mice. We independently generated Tl1a and Dr3 KO mice. For Tl1a KO, transcription and translation site and exon1 are deleted. For Dr3 KO, exons 2 to 5 are deleted. Using qPCR on ileal tissues, we show here that TL1A ko mice do not have TL1A expression and DR3 KO mice have no DR3 expression.

FIG. 3 illustrates, in accordance with various embodiments of the invention, chronic DSS-Induced colitis setup. The first model that we used to test the effect of TL1A and DR3 deficiency on intestinal inflammation is the chronic DSS model. In this model, WT, DR3 KO, and TL1A KO mice were treated with 4 cycles of 2% DSS over 4 wks. At the end of 4 weeks, mice were analyzed for differences in intestinal inflammation and immune function.

FIG. 4 illustrates, in accordance with various embodiments of the invention, Tl1a−/− but not Dr3−/− improves clinical DSS-induced colitis. Disease activity index (DAI) is comprised of weight loss, stool blood and consistency measurement. The higher the DAI, the worse the intestinal inflammation. Here we see that TL1A KO mice have lower DAI score compared to DR3 KO or WT, indicating that it has less clinical colitis compared to the other 2 groups.

FIG. 5 illustrates, in accordance with various embodiments of the invention, deficiency in Tl1a but not Dr3 reduce inflammation in the colon. The colon histology from 3 mouse groups was examined. We show here that WT and DR3 KO colons have increased inflammatory infiltrate, ulceration, and loss of crypt architecture as compared to TL1A KO. The histology scores are quantitated and we show here that TL1A deficiency leads to significantly reduced histological inflammation in the colon as compared to the other two groups.

FIG. 6 illustrates, in accordance with various embodiments of the invention, reduced tissue cell infiltrate with Tl1a−/− but not Dr3−/−. A marker of inflammation is increased cell infiltrate in the tissue. We therefore determined the cell number in MLN and in the colon and found that there is increased cell recovery from MLN and colon in WT and DR3 KO. We next quantitated the specific immune cell composition by flow cytometry and found that there is also an increase of total CD4 T cell, DC (dendritic cell), and Macrophages in either MLN or colon LPMC in WT and DR3 KO as compared to TL1A KO mice. This also indicates that there is reduced intestinal inflammation in the TL1A KO mice as compared to the other two groups.

FIG. 7 illustrates, in accordance with various embodiments of the invention, reduced IFNγ and IL17 producing CD4+ T cell in MLN of Tl1a−/− mice. We investigate if TL1A deficiency or DR3 deficiency lead to differences in proinflammatory cytokine expression. We show here that there is reduced IFNγ and IL17 producing cells from the MLN in the TL1A KO as compared to the other two groups.

FIG. 8 illustrates, in accordance with various embodiments of the invention, reduced T cell activation marker CD44 in LPMC of Tl1a−/− but not Dr3−/− mice. We next assessed whether there are any differences in the activation marker on CD4 T cells. We show here that the activation marker, CD44, is reduced in the colon in TL1A KO mice as compared to the other two groups.

FIG. 9 illustrates, in accordance with various embodiments of the invention, reduced activation marker CD80 in MLN of DC and MΦ of Tl1a−/− mice. We also assessed whether there are differences in the activation marker on dendritic cells and macrophages. We show here that there is significantly less dendritic cells and macrophages in the TL1A KO MLN that express the activation marker CD80 compared to WT and DR3 KO MLN.

FIG. 10 illustrates, in accordance with various embodiments of the invention, adoptive transfer colitis model setup. To confirm our findings that TL1A and DR3 have distinct phenotype and not model specific effect, we next used the adoptive transfer model. We transferred WT naïve CD4 T cells to RAG mice, and this has normal TL1A-DR3 expression. We transferred TL1A KO naïve CD4 T cells into TL1A KO RAG mice and the consequence is total lack of TL1A expression. Lastly, we transferred DR3 KO naïve CD4 T cells to DR3 KO RAG mice and the consequence is complete lack of DR3 expression. At the end of 6 weeks, mice were analyzed for differences in intestinal inflammation and immune function.

FIG. 11 illustrates, in accordance with various embodiments of the invention, Tl1a (not Dr3) deficiency prevents weight loss in adoptive transfer colitis model. The weight of 3 groups of mice was followed throughout the adoptive transfer model. We observed that there is reduced weight loss with TL1A KO mice as compared to the other two groups.

FIG. 12 illustrates, in accordance with various embodiments of the invention, deficiency in Tl1a but not Dr3 reduces inflammation in the colon. To determine whether there are differences in colonic inflammation, we examined colon histology from three mouse groups. We show here that WT and DR3 KO colon have increased inflammatory infiltrate and loss of crypt architecture as compared to TL1A KO. The histology scores are quantitated and we show here that TL1A deficiency leads to significantly reduced histological inflammation in the colon as compared to the other two groups.

FIG. 13 illustrates, in accordance with various embodiments of the invention, reduced T cells and DC in MLN of Tl1a (not Dr3) KO mice. We observed there is also increased cell recovery in the MLN of DR3 KO mice as compared to TL1A KO mice. Using flow cytometry, we also showed that there is increased total CD4 T cells and DCs in the DR3 KO as compared to TL1A KO MLN. Although there is no difference in the number of total cell and macrophage in the MLN between WT and TL1A KO, we observed that there are significant increased DCs in the MLN from WT as compared to TL1A KO mice.

FIG. 14 illustrates, in accordance with various embodiments of the invention, decreased Ifnγ-, Il17- and Il13-producing CD4 T cells in MLN of Tl1a KO mice. We assessed whether there are any differences in the cells that express proinflammatory cytokines in the MLN. We observed that there is significantly reduced IFNγ, IL17 and IL13 producing CD4 T cells from the MLN in the TL1A KO as compared to DR3 KO.

FIG. 15 illustrates, in accordance with various embodiments of the invention, reduced CD44 and CXCR3 CD4+ T cells in MLN and LPMC of Tl1a KO mice. To assess whether there are other differences in the CD4 T cells between the 3 groups, we determined the expression of the activation marker CD44 and trafficking marker CXCR3. We show that the expression of CD44 is reduced in TL1A KO as compared to WT and DR3 KO mice in both MLN and LPMC. There are also less TL1A KO CD4 T cells that express CXCR3 as compared to the other two groups. This may be one of the potential mechanisms for reduced inflammatory cell infiltrate in the colon of TL1A KO mice as compared to WT or DR3 KO mice.

FIG. 16 illustrates, in accordance with various embodiments of the invention, reduced activation marker CD80 on DC and MΦ in MLN of Tl1a KO mice. Expression of activation marker is also determined on dendritic cells and macrophages in the MLN. We showed that TL1A KO mice have fewer DC and macrophage that express CD80 as compared to WT and Dr3 KO.

FIG. 17 illustrates, in accordance with various embodiments of the invention, a summary of the results. In absence of TL1A, inflammation is suppressed in the intestine. A part of this seems due to decreased activation of T-cells and antigen presenting cells, and which lead to decreased proinflammatory cytokine suck as IFNγ, IL17 and IL13 and decreased trafficking marker such as CXCR3. In contrast to other tissues, modulation of TL1A and DR3 signaling leads to differential effects in the intestine. And this indicates an alternate DR3 ligand or alternate TL1A receptor in the intestine. Also, this indicates that TL1A blockade rather than DR3 blockade is a better approach when designing, screening and developing therapeutic treatments for IBD.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3rd ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein and as such can vary. The definitions and terminology used herein are provided to aid in describing particular embodiments, and are not intended to limit the claimed invention, because the scope of the invention is limited only by the claims.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

As used herein, the terms “treat,” “treatment,” “treating,” or “amelioration” when used in reference to a disease, disorder or medical condition, refer to both therapeutic treatment and prophylactic or preventative measures, wherein the object is to prevent, reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition even if the treatment is ultimately unsuccessful. Those in need of treatment include those already with the condition as well as those prone to have the condition or those in whom the condition is to be prevented.

“Beneficial results” or “desired results” may include, but are in no way limited to, lessening or alleviating the severity of the disease condition, preventing the disease condition from worsening, curing the disease condition, preventing the disease condition from developing, lowering the chances of a patient developing the disease condition, decreasing morbidity and mortality, and prolonging a patient's life or life expectancy. As non-limiting examples, “beneficial results” or “desired results” may be alleviation of one or more symptom(s), diminishment of extent of the deficit, stabilized (i.e., not worsening) state of intestinal inflammation and/or fibrosis, delay or slowing of intestinal inflammation and/or fibrosis, and amelioration or palliation of symptoms associated with intestinal inflammation and/or fibrosis.

“Diseases”, “conditions” and “disease conditions,” as used herein may include, but are in no way limited to any form of intestinal inflammation or intestinal inflammation-related condition, disease or disorder, for example, intestinal fibrosis, inflammatory bowel disease, Crohn's disease, ulcerative colitis, colitis, acute colitis, and chronic colitis.

As used herein, the term “administering,” refers to the placement an agent as disclosed herein into a subject by a method or route which results in at least partial localization of the agents at a desired site. “Route of administration” may refer to any administration pathway known in the art, including but not limited to aerosol, nasal, oral, transmucosal, transdermal, parenteral, enteral, topical or local. “Parenteral” refers to a route of administration that is generally associated with injection, including intracranial, intraventricular, intrathecal, epidural, intradural, intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions can be in the form of aerosol, lotion, cream, gel, ointment, suspensions, solutions or emulsions. In accordance with the present invention, “administering” can be self-administering. For example, it is considered as “administering” that a subject consumes a composition as disclosed herein.

The term “sample” or “biological sample” as used herein denotes a sample taken or isolated from a biological organism, e.g., a blood sample from a subject. Exemplary biological samples include, but are not limited to, cheek swab; mucus; whole blood, blood, serum; plasma; urine; saliva; semen; lymph; fecal extract; sputum; other body fluid or biofluid; cell sample; and/or tissue sample etc. The term also includes a mixture of the above-mentioned samples. The term “sample” also includes untreated or pretreated (or pre-processed) biological samples. In some embodiments, a sample can comprise one or more cells from the subject.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

A subject can be one who has been previously diagnosed with or identified as suffering from or having a condition in need of treatment (e.g., intestinal inflammation and/or fibrosis, IBD, and chronic colitis) or one or more complications related to the condition, and optionally, have already undergone treatment for the condition or the one or more complications related to the condition. Alternatively, a subject can also be one who has not been previously diagnosed as having a condition or one or more complications related to the condition. For example, a subject can be one who exhibits one or more risk factors for a condition or one or more complications related to the condition or a subject who does not exhibit risk factors. A “subject in need” of treatment for a particular condition can be a subject suspected of having that condition, diagnosed as having that condition, already treated or being treated for that condition, not treated for that condition, or at risk of developing that condition.

The term “statistically significant” or “significantly” refers to statistical evidence that there is a difference. It is defined as the probability of making a decision to reject the null hypothesis when the null hypothesis is actually true. The decision is often made using the p-value.

The terms “constitutive expression” and “constitutively expressing” describe that a gene is expressed continually or constantly. The terms “overexpression” and “overexpressing” describe that a gene is expressed at a level more than normal.

### Drug Screening Methods and Systems

Various embodiments of the present invention provide a model of a condition. In various embodiments, the condition is intestinal inflammation and/or fibrosis, inflammatory bowel disease, and/or chronic colitis.

Various embodiments of the present invention provide a method of identifying an agent as being therapeutic to a condition. In various embodiments, the method comprises: providing a model of the condition; administering the agent to the model; detecting one or more changes in the model to determine if the agent inhibits TL1A activity; and identifying the agent that is determined to inhibit TL1A activity as being therapeutic to the condition. In various embodiments, the condition is intestinal inflammation and/or fibrosis, inflammatory bowel disease, and/or chronic colitis. In various embodiments, the method further comprises detecting one or more changes in the model to determine if the agent inhibits DR3 activity, and identifying the agent that is determined to inhibit TL1A activity but not DR3 activity as being therapeutic to the condition.

Various embodiments of the present invention also provide a system for identifying an agent as being therapeutic to a condition. In various embodiments, the system comprises a model of the condition. In various embodiments, the system may further comprise one or more agents. In various embodiments, the system may further comprise one or more assays for determining if an agent inhibits TL1A activity. In various embodiments, the system may further comprise one or more assays for determining if an agent inhibits DR3 activity. In various embodiments, the condition is intestinal inflammation and/or fibrosis, inflammatory bowel disease, and/or chronic colitis.

In various embodiments, the one or more changes comprise a change in disease activity index (DAI), inflammation in the colon, cell infiltration (e.g., T cells, CD4+ T cells, antigen presenting cells (APCs), dendritic cells(DCs), and macrophages(MΦ) in MLN and LPMC), T-cell activation, APC activation, DC activation, MΦ activation, the amount of INFγ-producing CD4+ T cells, the amount of IL17-producing CD4+ T cells, the amount of IL13-producing CD4+ T cells, CD44 activation or expression, CD80 activation or expression, CXCR3 activation or expression, or body weight loss or gain, or a combination thereof.

In various embodiments, the model is a cell. In various embodiments, the model is a cell without genomic alteration. In some embodiments, the model is a wild type cell. In various embodiments, the model is a cell with genomic alteration. In accordance with the present invention, the cell may be from an animal, rodent or human. In various embodiments, the model is an animal. In various embodiments, the model is an animal without genomic alteration. In some embodiments, the model is a wild type animal. In various embodiments, the model is an animal with genomic alteration. In accordance with the present invention, the animal may be a rodent, mouse, rat, or guinea pig.

In some embodiments, the model is a quantity of cells overexpressing or constitutively expressing TL1A and/or DR3. In other embodiments, the model is an animal that has been injected with a quantity of cells overexpressing or constitutively expressing TL1A and/or DR3. In one embodiment, the animal is an immunodeficient rodent. In one embodiment, the cells are obtained from a transgenic animal with a transgene overexpressing or constitutively expressing TL1A and/or DR3 by a process, comprising: obtaining a sample comprising a population of cells from the transgenic animal; sorting the sample into a first sub-population of cells overexpressing or constitutively expressing TL1A and/or DR3, and a second sub-population of cells overexpressing or constitutively expressing neither TL1A or DR3; and separating the first sub-population from the second sub-population, thereby isolating the cells overexpressing or constitutively expressing TL1A and/or DR3.

In various embodiments, the model is a transgenic animal with a transgene overexpressing or constitutively expressing TL1A and/or DR3. In one embodiment, the TL1A and/or DR3 overexpression or constitutive expression is specific to a cell type. In some embodiments, the cell type is a myeloid cell. In certain embodiments, the myeloid cell is an antigen presenting cell (APC) or dendritic cell (DC). In other embodiments, the cell type is a lymphoid cell. In certain embodiments, the lymphoid cell is a T-cell. In various embodiments, the cell type expresses a fluorescent marker.

In various embodiments, the model is a gene knockout (KO) animal. As used herein, the term “gene knockout animal” refers to genetically modified animals in which one or more genes have been inactivated, disrupted, deleted, or “knocked out”. For example, a TL1A knockout animal has its TL1A gene inactivated, disrupted, deleted, or “knocked out”, and a DR3 knockout animal has its DR3 gene inactivated, disrupted, deleted, or “knocked out”. In various embodiments, the model is a gene knockout animal with TL1A and/or DR3 gene knocked out. In some embodiments, the model is a TL1A knockout animal. In some embodiments, the model is a DR3 knockout animal.

In various embodiments, the model expresses TL1A, DR3 and a fluorescent marker in about 30, 35, 40, 45, 50, 55, 60, 65, 70, 75, 80, 85, 90, 95, or 99% of all myeloid cells in a sample of myeloid cells isolated from the model. In various embodiments, the model expresses TL1A, DR3 and a fluorescent marker in about 30, 35, 40, 45, 50, 55, 60, 65, 70, 75, 80, 85, 90, 95, or 99% of all lymphoid cells in a sample of lymphoid cells isolated from the model. In various embodiments, the model expresses TL1A and a fluorescent marker in about 30, 35, 40, 45, 50, 55, 60, 65, 70, 75, 80, 85, 90, 95, or 99% of all myeloid cells in a sample of myeloid cells isolated from the model. In various embodiments, the model expresses DR3 and a fluorescent marker in about 30, 35, 40, 45, 50, 55, 60, 65, 70, 75, 80, 85, 90, 95, or 99% of all myeloid cells in a sample of myeloid cells isolated from the model. In various embodiments, the model expresses TL1A and a fluorescent marker in about 30, 35, 40, 45, 50, 55, 60, 65, 70, 75, 80, 85, 90, 95, or 99% of all lymphoid cells in a sample of lymphoid cells isolated from the model. In various embodiments, the model expresses DR3 and a fluorescent marker in about 30, 35, 40, 45, 50, 55, 60, 65, 70, 75, 80, 85, 90, 95, or 99% of all lymphoid cells in a sample of lymphoid cells isolated from the model.

In various embodiments, the model exhibits fibrostenosis, inflammation in the gastrointestinal (GI) tract, weight loss, and/or an increase in disease-activity index.

In various embodiments, the model has DSS-induced colitis. In various embodiments, the model has colitis induced by adoptive transfer. In various embodiments, the model is an adoptive transfer colitis model.

### Treatment Methods

Various embodiments of the present invention provide a method of treating, preventing, reducing the likelihood of having, reducing the severity of and/or slowing the progression of a condition in a subject. In various embodiments, the method comprises: providing an agent that inhibits TL1A activity; and administering a therapeutically effective amount of the agent to the subject, thereby treating, preventing, reducing the likelihood of having, reducing the severity of and/or slowing the progression of the condition in the subject. In various embodiments, the agent does not inhibit DR3 activity. In various embodiments, the agent is an anti-TL1A antibody or a functional fragment thereof. In some embodiments, the functional fragment of the anti-TL1A antibody is an antigen-binding fragment that specifically binds to TL1A.

In various embodiments, the condition is intestinal inflammation and/or fibrosis, inflammatory bowel disease, and/or chronic colitis. In various embodiments, the subject is a human. In various embodiments, the subject is a mammalian subject including but not limited to human, monkey, ape, dog, cat, cow, horse, goat, pig, rabbit, mouse and rat.

Typical dosages of an effective amount of the agent that inhibits TL1A activity can be in the ranges recommended by the manufacturer where known therapeutic molecules or compounds are used, and also as indicated to the skilled artisan by the in vitro responses in cells or in vivo responses in animal models. Such dosages typically can be reduced by up to about an order of magnitude in concentration or amount without losing relevant biological activity. The actual dosage can depend upon the judgment of the physician, the condition of the patient, and the effectiveness of the therapeutic method based, for example, on the in vitro responsiveness of relevant cultured cells or histocultured tissue sample, or the responses observed in the appropriate animal models. In various embodiments, the agent may be administered once a day (SID/QD), twice a day (BID), three times a day (TID), four times a day (QID), or more, so as to administer an effective amount of the agent to the subject, where the effective amount is any one or more of the doses described herein.

In various embodiments, the agent is administered at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/kg, or a combination thereof. In various embodiments, the agent is administered at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/m², or a combination thereof. In various embodiments, the agent is administered once, twice, three or more times. In various embodiments, the agent is administered about 1-3 times per day, 1-7 times per week, 1-9 times per month, or 1-12 times per year. In various embodiments, the agent is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years. Here, “mg/kg” refers to mg per kg body weight of the subject, and “mg/m²” refers to mg per m² body surface area of the subject. In certain embodiments, the agent is administered to a human. In various embodiments, the agent is an anti-TL1A antibody or a functional fragment thereof.

In various embodiments, the effective amount of the agent is any one or more of about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 μg/kg/day, or a combination thereof. In various embodiments, the effective amount of the agent is any one or more of about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 μg/m²/day, or a combination thereof. In various embodiments, the effective amount of the agent is any one or more of about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/kg/day, or a combination thereof. In various embodiments, the effective amount of the agent is any one or more of about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/m²/day, or a combination thereof. Here, “μg/kg/day” or “mg/kg/day” refers to μg or mg per kg body weight of the subject per day, and “μg/m²/day” or “mg/m²/day” refers to μg or mg per m² body surface area of the subject per day. In certain embodiments, the agent is administered to a human. In various embodiments, the agent is an anti-TL1A antibody or a functional fragment thereof.

In some embodiments, the agent may be administered at the prevention stage of a condition (i.e., when the subject has not developed the condition but is likely to or in the process to develop the condition). In other embodiments, the agent may be administered at the treatment stage of a condition (i.e., when the subject has already developed the condition).

In accordance with the invention, the agent may be administered using the appropriate modes of administration, for instance, the modes of administration recommended by the manufacturer for each of the agent. In accordance with the invention, various routes may be utilized to administer the agent of the claimed methods, including but not limited to intravenous, intraarterial, intramuscular, subcutaneous, intraperitoneal, aerosol, nasal, via inhalation, oral, transmucosal, transdermal, parenteral, implantable pump or reservoir, continuous infusion, enteral application, topical application, local application, capsules and/or injections. In various embodiments, the agent is administered topically, intravascularly, intravenously, intraarterially, intramuscularly, subcutaneously, intraperitoneally, intranasally, or orally.

### Pharmaceutical Compositions

Various embodiments of the present invention also provide a composition comprising an agent that inhibits TL1A activity. In various embodiments, the agent does not inhibit DR3 activity. In accordance with the present invention, a composition described herein may be used for treating, preventing, reducing the likelihood of having, reducing the severity of and/or slowing the progression of a condition in a subject. In various embodiments, the condition is intestinal inflammation and/or fibrosis, inflammatory bowel disease, and/or chronic colitis. In various embodiments, the subject is a human. In various embodiments, the agent is an anti-TL1A antibody or a functional fragment thereof. In some embodiments, the functional fragment of the anti-TL1A antibody is an antigen-binding fragment that specifically binds to TL1A.

In various embodiments, the agent in the composition is provided in mg agent per kilogram body weight of the subject; for example, about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/kg. In various embodiments, the agent in the composition is provided in mg agent per kilogram body weight of the subject; for example, about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/m². In certain embodiments, the composition is administered to a human.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal, parenteral, enteral, topical or local. “Parenteral” refers to a route of administration that is generally associated with injection, including intracranial, intraventricular, intrathecal, epidural, intradural, intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions can be in the form of aerosol, lotion, cream, gel, ointment, suspensions, solutions or emulsions. Methods for these administrations are known to one skilled in the art. In certain embodiments, the composition is formulated for intravascular, intravenous, intraarterial, intramuscular, subcutaneous, intraperitoneal, intranasal, or oral administration.

Preferred compositions will also exhibit minimal toxicity when administered to a mammal. In various embodiments, the composition is administered 1-3 times per day, 1-7 times per week, 1-9 times per month, or 1-12 times per year. In various embodiments, the composition is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years. In various embodiments, the composition may be administered once a day (SID/QD), twice a day (BID), three times a day (TID), four times a day (QID), or more, so as to administer an effective amount of the agent to the subject, where the effective amount is any one or more of the doses described herein.

In various embodiments, the pharmaceutical compositions according to the invention can contain any pharmaceutically acceptable excipient. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

In various embodiments, the pharmaceutical compositions according to the invention can contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving dry milling, mixing, and blending for powder forms; milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Before administration to patients, formulants may be added to the composition. A liquid formulation may be preferred. For example, these formulants may include oils, polymers, vitamins, carbohydrates, amino acids, salts, buffers, albumin, surfactants, bulking agents or combinations thereof.

Carbohydrate formulants include sugar or sugar alcohols such as monosaccharides, disaccharides, or polysaccharides, or water soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof “Sugar alcohol” is defined as a C4 to C8 hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used as long as the sugar or sugar alcohol is soluble in the aqueous preparation. In one embodiment, the sugar or sugar alcohol concentration is between 1.0 w/v % and 7.0 w/v %, more preferable between 2.0 and 6.0 w/v %.

Amino acids formulants include levorotary (L) forms of carnitine, arginine, and betaine; however, other amino acids may be added.

Polymers formulants include polyvinylpyrrolidone (PVP) with an average molecular weight between 2,000 and 3,000, or polyethylene glycol (PEG) with an average molecular weight between 3,000 and 5,000.

It is also preferred to use a buffer in the composition to minimize pH changes in the solution before lyophilization or after reconstitution. Most any physiological buffer may be used including but not limited to citrate, phosphate, succinate, and glutamate buffers or mixtures thereof. In some embodiments, the concentration is from 0.01 to 0.3 molar. Surfactants that can be added to the formulation are shown in EP Nos. 270,799 and 268,110.

Another drug delivery system for increasing circulatory half-life is the liposome. Methods of preparing liposome delivery systems are discussed in Gabizon et al., Cancer Research (1982) 42:4734; Cafiso, Biochem Biophys Acta (1981) 649:129; and Szoka, Ann Rev Biophys Eng (1980) 9:467. Other drug delivery systems are known in the art and are described in, e.g., Poznansky et al., DRUG DELIVERY SYSTEMS (R. L. Juliano, ed., Oxford, N.Y. 1980), pp. 253-315; M. L. Poznansky, Pharm Revs (1984) 36:277.

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

The compositions of the invention may be sterilized by conventional, well-known sterilization techniques. The resulting solutions may be packaged for use or filtered under aseptic conditions and lyophilized, the lyophilized preparation being combined with a sterile solution prior to administration. The compositions may contain pharmaceutically-acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and stabilizers (e.g., 1-20% maltose, etc.).

The pharmaceutical composition according to the invention can also be a bead system (e.g., pectin/zein hydrogel bead system) for delivering the therapeutic agent to the target cells (Yan F. et al., J Clin Invest. 2011 June; 121(6):2242-53).

### Kits of the Invention

In various embodiments, the present invention provides a kit for treating, preventing, reducing the severity of and/or slowing the progression of a condition in a subject. In various embodiments, the kit comprises: a quantity of an agent that inhibits TL1A activity; and instructions for using the agent to treat, prevent, reduce the likelihood of having, reduce the severity of and/or slow the progression of the condition in the subject. In various embodiments, the agent does not inhibit DR3 activity. In various embodiments, the agent is an anti-TL1A antibody or a functional fragment thereof. In some embodiments, the functional fragment of the anti-TL1A antibody is an antigen-binding fragment that specifically binds to TL1A.

The kit is an assemblage of materials or components, including at least one of the inventive compositions or components. Thus, in some embodiments the kit contains a composition including a drug delivery molecule complexed with a therapeutic agent, as described above.

The exact nature of the components configured in the inventive kit depends on its intended purpose. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to affect a desired outcome. Optionally, the kit also contains other useful components, such as, spray bottles or cans, diluents, buffers, pharmaceutically acceptable carriers, syringes, catheters, applicators (for example, applicators of cream, gel or lotion etc.), pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in assays and therapies. As used herein, the term “package” refers to a suitable solid matrix or material such as glass, plastic, paper, foil, and the like, capable of holding the individual kit components. Thus, for example, a package can be a glass vial used to contain suitable quantities of a composition as described herein. The packaging material generally has an external label which indicates the contents and/or purpose of the kit and/or its components.

Many variations and alternative elements have been disclosed in embodiments of the present invention. Still further variations and alternate elements will be apparent to one of skill in the art. Among these variations, without limitation, are the selection of constituent modules for the inventive compositions, and the diseases and other clinical conditions that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” As one non-limiting example, one of ordinary skill in the art would generally consider a value difference (increase or decrease) no more than 5% to be in the meaning of the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### EXAMPLES

The invention will be further explained by the following Examples, which are intended to be purely exemplary of the invention, and should not be considered as limiting the invention in any way. The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1 TL1A but not DR3 Deficiency Ameliorated Murine Models of Chronic Colitis

TL1A (a protein encoded by TNFSF15) is a TNF family member and an IBD associated gene that has been shown to modulate the adaptive immune response and exacerbates murine models of chronic colitis. Blocking Tl1a via neutralizing antibody can treat murine models of chronic colitis. DR3 is the only known receptor for TL1A. However, its role in gut mucosal inflammation has not been really shown.

We investigated the roles of Tl1a and Dr3 deficiency in murine models of chronic colitis. Chronic dextran sodium sulfate (DSS) and adoptive T-cell transfer models were used. Severity of intestinal inflammation was evaluated by disease activity index (DAI, composed of weight loss, stool blood, stool consistency) and histological analyses. Flow cytometry was used to determine immune cell infiltration and activation. ELISA was used for cytokine analysis. All mice used were littermates and mice of different genotypes were randomly co-housed.

Consistent with reports using neutralizing Tl1a antibodies in chronic murine colitis models, we found that Tl1a^{-/-} mice have significantly reduced DAI, gross gut inflammatory score, and histologic inflammation compared to WT mice that undergone chronic DSS treatment (Table 1). Interestingly, Dr3^{-/-} mice do not ameliorate murine DSS-induced colitis (Table 1). Tl1a deficiency but not Dr3 deficiency reduced MLN and LPMC cell infiltration and reduced the expression of pro-inflammatory cytokines (IFNγ and IL17, Table 1).

TABLE 1

| | | | | Tl1a^{-/-} vs |
|---|---|---|---|---|
| | WT | Tl1a^{-/-} | Dr3^{-/-} | Dr3^{-/-} |
| DAI | 5 | 3.6 | 5.9 | <0.01 |
| Gross Colitis | 2.1 | 1.4 | 2.5 | <0.001 |
| Histology | 13.5 | 9.4 | 13.6 | <0.001 |
| MLN (×10\hat{ }6) | 30.6 | 22.4 | 32.2 | <0.05 |
| LPMC | 1.19 | 0.83 | 1.68 | <0.05 |
| (×10\hat{ }6) | | | | |
| Ifnγ (%) | 12.3 | 7.0 | 12.6 | <0.05 |
| Il17 (%) | 3.41 | 1.99 | 3.49 | <0.05 |

We used the adoptive T-cell transfer model to confirm our findings in the chronic DSS-induced colitis model and to determine the potential cell types that mediate the effects of Tl1a-Dr3 signaling on gut mucosal inflammation. Tl1a deficiency in either transferred T cells (Tl1a^{-/-} to Rag1^{-/-}) or in recipient Rag1^{-/-}(WT to Tl1a^{-/-} Rag^{-/-}) mice have similar colitis disease activity as compared to control WT naïve T-cells to Rag1^{-/-} recipient (p=NS, data not shown). However, Tl1a deficiency in both adoptively transferred T-cells and recipient Rag1^{-/-} (Tl1a^{-/-} to Tl1a^{-/-}Rag^{-/-}) ameliorated colitis disease activity (Table 2). Dr3 deficiency in either T-cells (Dr3^{-/-}to RAG1^{-/-}), recipient Rag1^{-/-} mice (WT to Dr3^{-/-}Rag1^{-/-}), or both transferred T-cells and recipient RAG1 mice (Dr3^{-/-}to Dr3^{-/-}Rag1^{-/-}) did not ameliorate colitis compared to control (Table 2 and data not shown; p=NS).

TABLE 2

| | Donor | | | |
|---|---|---|---|---|
| | | | | Tl1a^{-/-} to Tl1a^{-/-}Rag1^{-/-} |
| | WT | Tl1a^{-/-} | Dr3^{-/-} | vs. |
| | Recipient | | | |
| | | Tl1a^{-/-} | | |
| | Rag1^{-/-} | Rag1^{-/-} | Dr3^{-/-}Rag1^{-/-} | Dr3^{-/-} to Dr3^{-/-}Rag1^{-/-} |
| DAI | 7.44 | 6.0 | 8.06 | <0.05 |
| Gross Colitis | 2.06 | 1.36 | 2.19 | <0.05 |
| Histology | 12.5 | 9.4 | 14.0 | <0.05 |
| MLN | 11.5 | 5.7 | 13.5 | <0.05 |
| (×10\hat{ }6) | | | | |
| LPMC | 1.27 | 1.14 | 1.49 | NS |
| (×10\hat{ }6) | | | | |
| Ifnγ (%) | 15.1 | 6.7 | 17.0 | NS |
| Il17 (%) | 9.8 | 9.0 | 11.5 | NS |

In conclusion, Tl1a deficiency, but not Dr3 deficiency, ameliorated two chronic murine colitis models. Our results highlight TL1A, but not DR3, as the therapeutic target of choice in IBD.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

1. A method of identifying an agent as being therapeutic to a condition, comprising:
providing a model of the condition;
administering the agent to the model;
detecting one or more changes in the model to determine if the agent inhibits TL1A activity; and
identifying the agent that is determined to inhibit TL1A activity as being therapeutic to the condition.

2. The method of claim 1, wherein the condition is intestinal inflammation and/or fibrosis, inflammatory bowel disease, and/or chronic colitis.

3. The method of claim 1, wherein the model is a quantity of cells overexpressing or constitutively expressing TL1A and/or DR3.

4. The method of claim 3, wherein the cells are obtained from a transgenic animal with a transgene overexpressing or constitutively expressing TL1A and/or DR3 by a process, comprising:
obtaining a sample comprising a population of cells from the transgenic animal;
sorting the sample into a first sub-population of cells overexpressing or constitutively expressing TL1A and/or DR3, and a second sub-population of cells overexpressing or constitutively expressing neither TL1A or DR3; and
separating the first sub-population from the second sub-population, thereby isolating the cells overexpressing or constitutively expressing TL1A and/or DR3.

5. The method of claim 1, wherein the model is an animal that has been injected with a quantity of cells overexpressing or constitutively expressing TL1A and/or DR3.

6. The method of claim 5, wherein the animal is an immunodeficient rodent.

7. The method of claim 1, wherein the model is a transgenic animal with a transgene overexpressing or constitutively expressing TL1A and/or DR3.

8. The method of claim 7, wherein the TL1A and/or DR3 overexpression or constitutive expression is specific to a cell type.

9. The method of claim 8, wherein the cell type is a myeloid cell.

10. The method of claim 9, wherein the myeloid cell is an antigen presenting cell (APC) or dendritic cell (DC).

11. The method of claim 8, wherein the cell type is a lymphoid cell.

12. The method of claim 11, wherein the lymphoid cell is a T-cell.

13. The method of claim 8, wherein the cell type expresses a fluorescent marker.

14. The method of claim 1, wherein the model expresses TL1A, DR3 and a fluorescent marker in about 30, 35, 40, 45, 50, 55, 60, 65, 70, 75, 80, 85, 90, 95, or 99% of all myeloid cells in a sample of myeloid cells isolated from the model.

15. The method of claim 1, wherein the model expresses TL1A, DR3 and a fluorescent marker in about 30, 35, 40, 45, 50, 55, 60, 65, 70, 75, 80, 85, 90, 95, or 99% of all lymphoid cells in a sample of lymphoid cells isolated from the model.

16. The method of claim 1, wherein the model exhibits fibrostenosis, inflammation in the gastrointestinal (GI) tract, weight loss, and/or an increase in disease-activity index.

17. The method of claim 1, wherein the model has DSS-induced colitis.

18. The method of claim 1, wherein the model has colitis induced by adoptive transfer.

19. The method of claim 1, wherein the model a gene knockout animal with TL1A and/or DR3 gene knocked out.

20. A model of a condition, wherein the model is a quantity of cells overexpressing or constitutively expressing TL1A and/or DR3; or an animal that has been injected with a quantity of cells overexpressing or constitutively expressing TL1A and/or DR3; or a transgenic animal with a transgene overexpressing or constitutively expressing TL1A and/or DR3; or a gene knockout animal with TL1A and/or DR3 gene knocked out.

21. A method of treating, preventing, reducing the likelihood of having, reducing the severity of and/or slowing the progression of a condition in a subject, comprising:
providing an agent that inhibits TL1A activity; and
administering a therapeutically effective amount of the agent to the subject, thereby treating, preventing, reducing the likelihood of having, reducing the severity of and/or slowing the progression of the condition in the subject.

22. A composition comprising an agent that inhibits TL1A activity.
